(19) United States
(12) Patent Application Publication
(10) Pub. No.: US 2024/0206458 A1
(43) Pub. Date: Jun. 27, 2024

(54) METHOD FOR LARGE-SCALE BANKING OF HUMAN PLURIPOTENT STEM CELLS AND PRODUCTS DERIVED THEREOF

(71) Applicant: Novo Nordisk A/S, Bagsvaerd (DK)

(72) Inventors: Arvind Pradip, Frederiksberg (DK); Janne Jensen, Skodsborg (DK); Michael Wagner Christiansen, Lynge (DK); Amna Magzoub, Copenhagen (DK)

(21) Appl. No.: 18/576,859

(22) PCT Filed: Jul. 12, 2022
(86) PCT No.: PCT/EP2022/069424

§ 371 Date: Jan. 5, 2024

(30) Foreign Application Priority Data
Jul. 13, 2021 (EP) 21185214.0

(51) Int. Cl. A01N 1/02

(52) U.S. Cl. CPC A01N 1/0284; A01N 1/0252

(57) ABSTRACT

The present invention relates to a method for establishing a cell bank, comprising the steps of providing a batch of cell suspension comprising stem cells or stem cell-derived cells, and a cryopreservation medium, maintaining the batch of cell suspension at a temperature below 15° C., and transferring an amount of the cell suspension from the batch of cell suspension into one or more storage containers, wherein establishing the cell bank takes at least (30) minutes.

### TECHNICAL FIELD

The present invention relates generally to the field of stem cells, such as human pluripotent stem cells, and stem cell-derived cell products. Specifically, methods are provided for establishing cell banks from human pluripotent stem cells (hPSCs), such as undifferentiated hPSC banks and hPSC-derived drug substance and drug products.

### BACKGROUND

The prospect of using hPSCs in the treatment of various conditions seems very promising. As efforts are being made to bring viable cell-based treatments closer to the market it becomes increasingly relevant to consider manufacturing at large scale and production settings. For some indications an estimated 10⁸ cells are needed per treatment. The production of a stem cell-based product typically uses the differentiation of hPSCs as a starting point. Cell lines may be derived from early stage human embryos or by induced pluripotency of somatic cells. The resulting cell line is then cultured for expansion and establishment of a cell bank, wherein the cells are cryopreserved for storage until they undergo further expansion and/or differentiation into a specific cell-type product.

Over the years significant improvements have been made in cryopreservation of hPSCs. The fundamental cryopreservation principles originate from other mammalian cell types, where the procedures rely on chemical in cryopreservation medium like disaccharides sucrose, maltose, trehalose and others with and without DMSO.

The main cause of cell death is not the long-term storage by itself at low temperatures, but events that happen to the cells over glass-transition temperature (T_{g}). The intracellular crystallization during freezing and re-crystallization upon warming is known to cause most damage to cells apart from other known causes like toxicity, cell shrinkage, osmolality imbalance etc. To counter these detrimental events to cells and especially to hPSCs, different cryopreservation methods (vitrification, slow cooling, rapid thaw, etc.) and cryopreservation mediums have been developed.

For therapeutic cell production, long term stability of cells and a methodology allowing to generate large cell banks is crucial for success. It is therefore an object of the present invention to improve current methods for establishing cell banks with high cell viability whilst complying with the requirements of a therapeutic drug product including good cell culture practice (GCCP) and current good manufacturing practice (cGMP), in particular addressing the issues arising from establishing cell banks that takes an increased amount of time due to preparation and/or large-scale banking.

### SUMMARY

The object as outlined above is achieved by the aspects of the present invention. In addition, the present invention may also solve further problems, which will be apparent from the disclosure of the exemplary embodiments.

In a first aspect of the present invention is provided a method for establishing a cell bank, comprising the steps of providing a batch of cell suspension comprising stem cells or stem cell-derived cells, and a cryopreservation medium, maintaining the batch of cell suspension at a temperature below 15° C., and transferring an amount of the cell suspension from the batch of cell suspension into one or more storage containers to establish the cell bank, wherein establishing the cell bank takes at least 30 minutes. The present inventors have realized that establishing a large cell bank by filling a multitude of storage containers, such as vials, with cells requires an extended amount of time during which the viability of the cells in a cryopreservation medium decreases significantly. The same applies when establishing cell banks, wherein the batch of cell suspension requires additional time of preparation. By maintaining the batch of cell suspension at a temperature of below 15° C. for the entire process of establishing the cell bank the quality and cell count of each vial will be improved.

In a further aspect is provided a cooling apparatus for cooling a batch of cell suspension during preparation of a cell bank comprising a housing comprising an outer wall, a first compartment adapted to receive a refrigerant, and a second compartment adapted to hold a container with the batch of cell suspension, wherein the first and second compartment are separated by a heat conducting element, whereby the refrigerant is capable of cooling the batch of cell suspension when the refrigerant is loaded into the first compartment, and the container with the batch of cell suspension is loaded into the second compartment. The present inventors have designed a cooling apparatus suitable for maintaining a batch of cell suspension at a temperature below 15° ° C. while establishing a cell bank, where the use of the cooling apparatus facilitates compliance with the strict requirements of GCCP and cGMP. The cooling apparatus is designed as such to not generate any particles during operation so this apparatus can be handled within clean room area classification of grade A (alternatively ISO designation 5) complying to requirements of EU GMP Guidance Annex 1: Manufacturing of Sterile Medicinal Products and US Food and Drug Administration's (FDA's) Guidance for Industry: Sterile Drug Products Produced by Aseptic Processing Current Good Manufacturing Practice.

Accordingly, a further aspect of the present invention relates to the use of the cooling apparatus for the preparation of a cell bank from a batch of cell suspension, comprising maintaining the temperature of the batch of cell suspension at below 15° C., preferably 0-12° C., more preferably 0-10° C., more preferably 0-8° C., more preferably 2-8° C., more preferably 2-6° C., more preferably 3-5° C., even more preferably about 4° C.

In a final aspect is provided a method of cooling a batch of cell suspension comprising the steps of providing a cooling apparatus as described above, loading a refrigerant into the first compartment of the cooling apparatus, wherein the temperature of the refrigerant is suitable for maintaining a temperature of the batch of cell suspension below 15° C., loading a flask containing the batch of cell suspension into the second compartment of the cooling apparatus, and maintaining a temperature of the batch of cell suspension at below 15° C.

### BRIEF DESCRIPTION OF DRAWINGS

FIG. 1 shows the housing of the cooling apparatus with temperature conducting elements separating the first and second compartment.

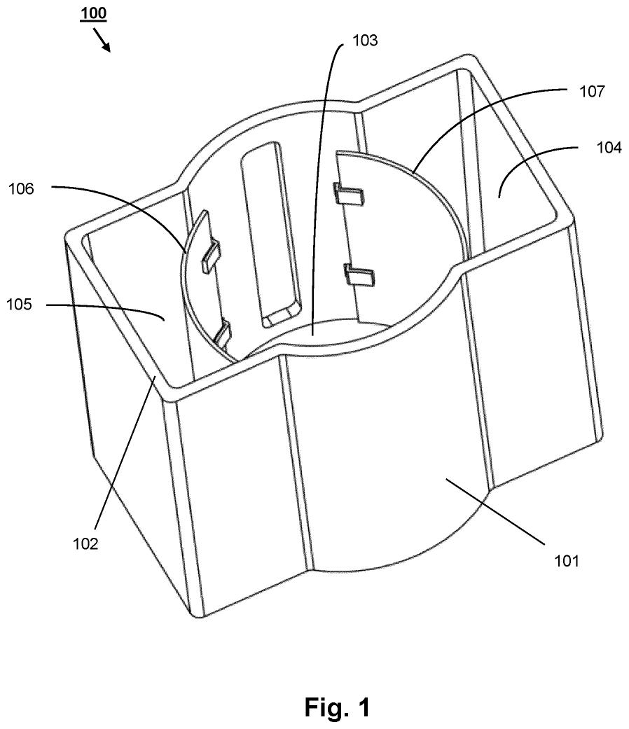

FIG. 2 shows a top view of the cooling apparatus with heat conducting elements separating the first and second compartment.

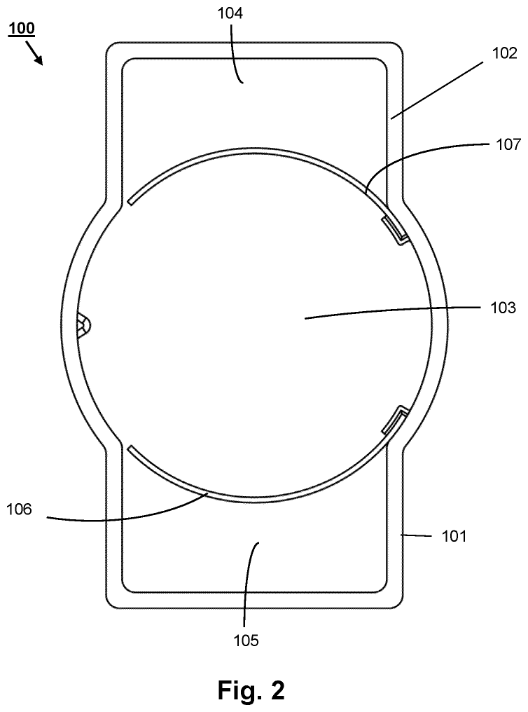

FIG. 3 illustrates the partly disassembled cooling apparatus with a lid, a heat conducting element and the housing having one heat conducting element mounted to separate the first and second compartment.

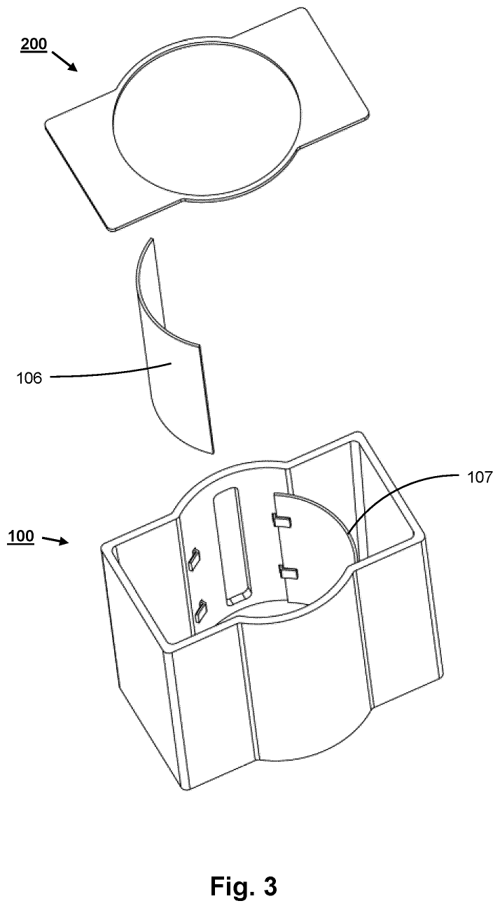

FIG. 4 shows viability by Tryphan blue exclusion method of hESCs over different exposure time to cryopreservation medium (specifically, STEM-CELLBANKER) at room temperature.

FIG. 5 shows an experiment the viability by Tryphan blue exclusion method of hESCs over increasing exposure time to cryopreservation medium (specifically, STEM-CELLBANKER) when cell suspension is maintained at about 4° C.

FIG. 6 shows temperature measurement curves of the medium in a container in the cooling apparatus.

FIG. 7 shows % viability of hESC derived beta-like cells measured by NC-202 for different holding time of cryopreservation medium (Like, StemcellBanker) at room temperature and around 4 degrees Celsius. The average % viability drops from 82 to 66 at RT compared to 89 to 85 at 4° C. (5 min to 240 min). 6-8 vials thawed for each time point and by 3 different operators. 2 way ANOVA Sidak's multiple comparison test done to compare RT vs 4° C.

FIG. 8 shows flow cytometry dot plots comparing percentage of cells expressing NKX6.1 and Islet-1 marker. 49.5% co-expresses the two markers denoting beta cells.

FIG. 9 shows % viability of hESC derived RPE cells measured by NC-202 for different holding time of cryopreservation medium (Like, StemcellBanker) at room temperature and around 4 degrees Celsius. The % viability drops from 95 to 81 at RT compared to 95 to 89% at 4° C. (0 min to 240 min). 8-10 vials thawed for each time point and by 3 different operators. 2 way ANOVA Sidak's multiple comparison test done to compare RT vs 4° C.

### DESCRIPTION

Unless otherwise stated, all technical and scientific terms used herein have the same meanings as commonly understood by one of ordinary skill in the art to which this invention belongs. The practice of the present invention employs, unless otherwise indicated, conventional methods of chemistry, biochemistry, biophysics, molecular biology, cell biology, genetics, immunology and pharmacology, known to those skilled in the art.

It is noted that all headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed.

### General Definitions

Throughout this application the terms “method” and “protocol” when referring to processes for differentiating cells may be used interchangeably. As used herein, “a” or “an” or “the” can mean one or more than one. Unless otherwise indicated in the specification, terms presented in singular form also include the plural situation. As used herein, “and/or” refers to and encompasses any and all possible combinations of one or more of the associated listed items, as well as the lack of combinations when interpreted in the alternative (“or”). Moreover, the present invention also contemplates that in some embodiments of the invention, any feature or combination of features set forth herein can be excluded or omitted.

Hereinafter, the methods according to the present invention are described in more detail by non-limiting embodiments and examples. A method is provided for establishing large stem cell banks. Accordingly, the method take offset in the use of stem cells.

By “stem cell” is to be understood a cell having differentiation potency and proliferative capacity (particularly self-renewal competence) but maintaining differentiation potency. The stem cell includes subpopulations such as pluripotent stem cell, multipotent stem cell, unipotent stem cell and the like according to the differentiation potency. As used herein, the term “pluripotent stem cell” (PSC) refers to a stem cell capable of being cultured in vitro and having a potency to differentiate into any cell lineage belonging to three germ layers (ectoderm, mesoderm, endoderm) and/or extraembryonic tissue (pluripotency). As used herein, the term “multipotent stem cell” means a stem cell having a potency to differentiate into plural types of tissues or cells, though not all kinds. As used herein, the term “unipotent stem cell” means a stem cell having a potency to differentiate into a particular tissue or cell. A pluripotent stem cell can be induced from fertilized egg, clone embryo, germ stem cell, stem cell in a tissue, somatic cell and the like. Examples of the pluripotent stem cell (PSC) include embryonic stem cell (ESC), EG cell (embryonic germ cell), induced pluripotent stem cell (iPSC) and the like. Muse cell (Multi-lineage differentiating stress enduring cell) obtained from mesenchymal stem cell (MSC), and GS cell produced from reproductive cell (e.g., testis) are also encompassed in the pluripotent stem cell. As used herein, the term “induced pluripotent stem cell” (also known as iPS cells or iPSCs) means a type of pluripotent stem cell that can be generated directly from adult cells. As used herein, the term “embryonic stem cell” (also known as hES cells or hESCs) means a type of pluripotent stem cell that have been derived from either a single blastomere or the inner cell mass of a blastocyst, or from parthenotes (as described in e.g. WO 2003/046141). Embryonic stem cells are available from given organizations and are also commercially available. Preferably, the methods and products of the present invention are based on hPSCs, i.e. stem cells derived from either induced pluripotent stem cells or embryonic stem cells, including parthenotes.

When referring to viability measurements by “NC-202” is meant The NucleoCounter® NC-202™ automated cell counter (NucleoCounter® NC-202™ Leading Automated Cell Counter System (chemometec.com)).

As used herein, the term “RPE cell” means retinal pigment epithelium cell.

### Method for Establishing a Cell Bank

In a first aspect of the present application is provided a method for establishing a cell bank, comprising the steps of a) providing a batch of cell suspension comprising stem cells or stem cell-derived cells, and a cryopreservation medium, b) maintaining the batch of cell suspension at a temperature below 15° C., and c) transferring an amount of the cell suspension from the batch of cell suspension into one or more storage containers to establish the cell bank, wherein establishing the cell bank takes at least 30 minutes.

As used herein, the term “cell bank” refers to aliquots of a single pool of cells that typically has been prepared from an individual source derived under defined conditions, dispensed into multiple containers, and stored under defined conditions. It may further be a pool of different cells types.

As used herein, the term “cell suspension” means that cells are free-floating in a liquid medium thus allowing for maintaining a substantially homogeneous concentration of cells, wherein aliquots of the liquid medium comprising cells can be transferred. The cells in the cell suspension may be single cells or cell aggregates/spheroids, and with or without biomaterials.

As used herein, the term “batch of cell suspension” refers to the cell suspension which is to be transferred into one or more storage containers.

As used herein, the term “stem cell-derived cell” means a stem cell which has been differentiated. The term “differentiation” in respect to pluripotent stem cells refers to the process wherein cells progress from an undifferentiated state to a specific differentiated state, i.e. from an immature state to a less immature state or to a mature state. Changes in cell interaction and maturation occur as cells lose markers of undifferentiated cells or gain markers of differentiated cells. Loss or gain of a single marker can indicate that a cell has “matured or fully differentiated”.

By “providing a batch of cell suspension” in step a) is meant obtaining a batch of cell suspension by any suitable means. In an embodiment, the batch of cell suspension is provided by formulating the stem cells or stem cell-derived cells with a cryopreservation medium. In an embodiment, the concentration of the cells in the batch of cell suspension is from about 1×10⁵ cells/ml to about 1×10⁸ cells/ml. In an embodiment, the stem cells are PSCs. In a further embodiment, the stem cells are hPSCs. The stem cells may be provided by any suitable method as referred to above, e.g. the stem cells may be provided by deriving from human embryonic stem cells. A person skilled in the art will recognize suitable methods for obtaining or providing stem cells, which may include deriving stem cells from blastomeres or the inner cell mass of a blastocyst. As used herein a “cryopreservation medium” means a liquid medium composition suitable for preserving cells during freezing. In an embodiment, the cryopreservation medium comprises dimethyl sulfoxide (DMSO). In a preferred embodiment, the cryopreservation medium is chemically defined, xeno-free, and GMP grade. Suitable cryopreservation media are commercially available, such as STEM-CELLBANKER.

In step b) the batch of cell suspension is maintained at a temperature of below 15° C. In an embodiment, the temperature is maintained at 0-12° C., preferably 0-11° C., preferably 0-10° C., preferably 0-8° C., more preferably 2-8° C., more preferably 2-6° C., more preferably 3-5° C., even more preferably about 4° C. In an embodiment, the temperature is maintained below 15° C., 14° C., 13° C., 12° C., 11° C., 10° C., 9° C., 8° C., 7° C., 6° C., 5° C., or 4° C., preferably below 8° C. However, the temperature of the batch of cell suspension should not be below the freezing point of the cell suspension. A skilled person will readily be able to establish the freezing point of the cell suspension. In an embodiment, the temperature is maintained above the freezing point of the cell suspension. In a further embodiment, the temperature is maintained above 0° C., 1° C., 2° ° C., or 3° C. The temperature may be maintained at said temperature by any suitable means, such as by cooling the container with the batch of cell suspension or by cooling the surroundings wherein the cell bank is established. In an embodiment, the batch of cell suspension in step b) is subjected to agitation, such as shaking or stirring. The batch of cell suspension may be agitated by any suitable means such as by magnetic stirring. Agitation may facilitate a homogeneous cell suspension thereby ensuring a substantially even concentration of cells transferred into each vial in step c).

In step c) the an amount of the cell suspension from the batch of cell suspension is transferred to one or more storage containers. In an embodiment, an amount of the cell suspension is transferred to at least 10, 50, 100, 200, 300, 400, 500, 600, 700, 800, 900, or 1000 storage containers. As used herein the term “storage container” refers to a container suitable for storing the cells. Any container suitable for storing the cells may be used, such as a glass or plastic vessel or bottle or bag. In an embodiment, the storage container is a vial or a cryobag. As used herein, the terms “amount” and “aliquot” may be used interchangeably and refer to a volume smaller than the initial volume of the cell suspension. Any suitable method for transferring an amount of the cell suspension may be used. The transfer is typically carried out manually by pipette, but may also be automated with robotic assistance. In an embodiment, the amount of cell suspension transferred into each vial is from 0.25 to 1000 ml. In an embodiment, an amount of about 1 ml is transferred to a 2 ml cryovial. In an embodiment, an amount of about 20 ml is transferred to a 50 ml cryobag. In an embodiment, the volume of the batch of cell suspension is at least 25 ml, 50 ml, 100 ml, 200 ml, 300 ml, 400 ml, 500 ml, 600 ml, 700 ml, 800 ml, 900 ml, or 1 L.

In an embodiment, establishing the cell bank takes at least one hour, at least two hours, or at least three hours. The time in which the cell suspension is maintained at the temperature depends on the time it takes to establish the cell bank, i.e. the time it takes to prepare and/or transfer an amount of the cell suspension to each of the number of vials. It should be readily understood that the step of transferring aliquots of cells suspension to storage containers is carried out while maintaining the remaining batch of cell suspension at said temperature. Once an amount of the cell suspension is transferred to a storage container it is no longer necessarily maintained at said temperature.

In an embodiment, the method further comprises step d) of freezing each of the storage containers immediately following transfer of the amount of the cell suspension. Further to the findings by the present inventors it should readily be appreciated that the individual storage containers following transfer of an aliquot of cell suspension should not be left at e.g. room temperature while establishing the remaining cell bank. By the term “immediately” is meant that the cell suspension transferred into a vial is subjected to freezing within less than 10 minutes after transfer, preferably less than 8, 6, 4, or 2 minutes after transfer. A person skilled in the art will recognize suitable methods for freezing as well as recommended freezing temperatures for storage.

In an embodiment, the method is for increasing the viability of the cells in the cell suspension.

In an embodiment, the stem cells or stem cell-derived cells are single cells or aggregates. In another embodiment, the batch of cell suspension further comprises biomaterial.

In an embodiment, the batch of cell suspension in step a) is formulated in a cell culture spinner bottle, and the cell culture spinner bottle is placed in a cooling apparatus according to the present invention.

### Cooling Apparatus

In another aspect is provided a cooling apparatus 100 for cooling a batch of cell suspension during preparation of a cell bank comprising: a housing 101 comprising an outer wall 102, a first compartment 104, 105 adapted to receive a refrigerant, and a second compartment 103 adapted to hold a container with the batch of cell suspension, wherein the first 104, 105 and second 103 compartment are separated by a heat conducting element 106, 107, whereby the refrigerant is capable of cooling the batch of cell suspension when the refrigerant is loaded into the first compartment 104, 105, and the container with the batch of cell suspension is loaded into the second compartment 103.

In an embodiment, the housing 101 is made from nylon. In an embodiment, the housing 101 is 3D printed using SLS printing resulting in a nylon material, which is an inert and robust material that can handle frequent sanitization with EtOH/IPA.

In an embodiment, the cooling apparatus 100 comprises two first compartments 104, 105 on opposite sides of the second compartment 103. It follows that in this embodiment, both first compartments 104, 105 are separated by a heat conducting element 106, 107. This provides for a more evenly distribution of the cooling of the batch of cell suspension. The heat conducting element 106, 107 may be any suitable material for conducting heat. In an embodiment, the heat conducting element 106, 107 is a metal block, preferably a stainless steel block. In an embodiment, the heat conducting element 106, 107 is curved to fit closely to the container with the batch of cell suspension.

In an embodiment, the cooling apparatus further comprises a lid 200 to cover the one or more first compartments 104, 105.

As used herein, the term “refrigerant” means a substance suitable for cooling. In an embodiment, the refrigerant is in a sealed container. In a preferred embodiment, the refrigerant is in a bag, such as a gel pack. In an embodiment, the refrigerant is pre-cooled to a temperature below −20° C.

As used herein, the term “container” in reference to the cooling apparatus refers to a container comprising the batch of cell suspension which is to be distributed into smaller storage containers, such as vials. In an embodiment, the container with the batch of cell suspension has a volume of from 50 ml to 10 L, preferably 1 L. In an embodiment, the container with the batch of cell suspension is a cell culture spinner bottle.

In a preferred embodiment, the cooling apparatus is GMP compliant. Specifically, GMP compliant for a cleanroom class A environment.

### Method of Cooling Cell Suspension

It follows that another aspect of the present invention relates to a method of cooling a batch of cell suspension comprising the steps of i) providing a cooling apparatus 100 as described above, ii) loading a refrigerant into the first compartment 104, 105 of the cooling apparatus 100, wherein the temperature of the refrigerant is suitable for maintaining a temperature of the batch of cell suspension below 15° C., iii) loading a container containing the batch of cell suspension into the second compartment 103 of the cooling apparatus 100, and iv) maintaining a temperature of the batch of cell suspension below 15° C.

In an embodiment, the refrigerant is a cooling pack. As used herein, the term “cooling pack” means container, such as a plastic bag, filled with a refrigerant. In an embodiment, the temperature of the refrigerant is from −20 to −80° C. when loaded into the first compartment 104, 105. In an embodiment, the temperature of the cell suspension is maintained at 0-15° C., preferably 0-12° ° C., preferably 0-10° C., preferably 0-8° C., more preferably 2-8° C., more preferably 2-6° C., more preferably 3-5° C., even more preferably about 4° C.

In a preferred embodiment, the method is GMP compliant.

### Use of Cooling Apparatus

Another aspect of the present invention relates to the use of a cooling apparatus as described hereinbefore for the preparation of a cell bank from a batch of cell suspension, comprising maintaining the temperature of the batch of cell suspension below 15° C., preferably 0-12° C., preferably 0-10° C., more preferably 0-8° C., more preferably 2-8° C., more preferably 2-6° C., more preferably 3-5° C., even more preferably about 4° C. In a preferred embodiment, the cells are stem cells or derived from stem cells. In an even more preferred embodiment, the stem cells are human pluripotent stem cells (hPSCs).

In an embodiment, the refrigerant is replaced as required to maintain the low temperature of the batch of cell suspension.

### PARTICULAR EMBODIMENTS

The aspects of the present invention are now further described by the following non-limiting embodiments:
1. A cooling apparatus (100) for cooling a batch of cell suspension during preparation of a large cell bank comprising: a housing (101) comprising an outer wall (102), a first compartment (104) adapted to receive a refrigerant, and a second compartment (103) adapted to hold a container with the batch of cell suspension, wherein the first (104) and second (103) compartment are separated by a heat conducting element (106), whereby the refrigerant is capable of cooling the batch of cell suspension when the refrigerant is loaded into the first compartment (104), and the container with the batch of cell suspension is loaded into the second compartment (103).
2. The cooling apparatus according to the preceding embodiment, wherein the housing (101) is made from nylon.
3. The cooling apparatus according to any one of the preceding embodiments, wherein the heat conducting element (106) is a metal block, preferably a stainless steel block.
4. The cooling apparatus according to any one of the preceding embodiments, wherein the refrigerant is in a sealed bag, such as a gel pack.
5. The cooling apparatus according to any one of the preceding embodiments, wherein the container with the batch of cell suspension has a volume of from 50 ml to 10 L, preferably 1 L.
6. The cooling apparatus according to any of the preceding embodiments, wherein the container with the batch of cell suspension is a cell culture spinner bottle.
7. The cooling apparatus according to any one of the preceding embodiments, wherein the cooling apparatus is GMP compliant.
8. The cooling apparatus according to any one of the preceding embodiments, wherein the cooling apparatus comprises two first compartments (104, 105) on opposite sides of the second compartment (103).
9. The cooling apparatus according to embodiment 8, wherein each of the two first compartments (104, 105) are separated from the second compartment (103) by a heat conducting element (106, 107).
10. The cooling apparatus according to any one of the preceding embodiments, further comprising a lid (200) to cover the one or more first compartments (104, 105).
11. A method for establishing a cell bank, comprising the steps of:
   a. providing a batch of cell suspension comprising stem cells or stem cell-derived cells, and a cryopreservation medium,
   b. maintaining the batch of cell suspension at a temperature below 15° ° C., and
   c. transferring an amount of the cell suspension from the batch of cell suspension into one or more storage containers to establish the cell bank,
   wherein establishing the cell bank takes at least 30 minutes.
12. The method according to embodiment 11, comprising the additional step of:
   d. freezing each of the storage containers immediately following transfer of the amount of the cell suspension.
13. The method according to any one of embodiments 11 and 12, wherein an amount of the cell suspension is transferred to at least 10, 50, 100, 200, 300, 400, 500, 600, 700, 800, 900, or 1000 storage containers.
14. The method according to any one of embodiments 11 to 13, wherein the temperature is maintained at 0-15° C., preferably 0-12° C., preferably 0-11° C., preferably 0-10° C., preferably 0-8° C., more preferably 2-8° C., more preferably 2-6° C., more preferably 3-5° C., even more preferably about 4° C.
15. The method according to any one of embodiments 11 to 14, wherein the temperature is maintained below 15° C., 14° C., 13° C., 12° C., 12° C., 11° C., 10° C., 9° C., 8° C., 7° C., 6° C., 5° C., or 4° C., preferably below 12° C., more preferably below 8° C.
16. The method according to any one of embodiments 11 to 15, wherein the temperature is maintained above the freezing point of the cell suspension.
17. The method according to any one of embodiments 11 to 16, wherein the temperature is maintained above 0° C., 1° C., 2° C., or 3° C.
18. The method according to any one of embodiments 11 to 17, wherein establishing the cell bank takes at least one hour, at least two hours, or at least three hours.
19. The method according to any one of embodiments 11 to 18, wherein the storage containers are vials and/or cryobags.
20. The method according to any one of embodiments 11 to 19, wherein the method is for increasing the viability of the cells.
21. The method according to any one of embodiments 11 to 20, wherein the concentration of the cells in the batch of cell suspension is from about 1×10⁵ cells/ml to about 1×10⁸ cells/ml.
22. The method according to any one of embodiments 11 to 21, wherein the cryopreservation medium comprises dimethyl sulfoxide (DMSO).
23. The method according to any one of embodiments 11 to 22, wherein the cryopreservation medium is STEM-CELLBANKER.
24. The method according to the any one of embodiments 11 to 23, wherein the stem cells are pluripotent stem cells (PSCs).
25. The method according to embodiment 24, wherein the stem cells are human pluripotent stem cells (hPSCs).
26. The method according to any one of embodiments 11 to 25, wherein the stem cells or stem cell-derived cells are single cells or aggregates.
27. The method according to any one of embodiments 11 to 26, wherein the stem cell-derived cells are beta like cells, cardiomyocytes, or RPE cells.
28. The method according to any one of embodiments 11 to 27, wherein the cell suspension further comprises biomaterial.
29. The method according to any one of embodiments 11 to 28, wherein the volume of the batch of cell suspension is at least 25 ml, 50 ml, 100 ml, 200 ml, 300 ml, 400 ml, 500 ml, 600 ml, 700 ml, 800 ml, 900 ml, or 1 L.
30. The method according to any one of embodiments 11 to 29, wherein the amount of cell suspension transferred into each storage container is from 0.25 to 1000 ml.
31. The method according to any one of embodiments 11 to 30, wherein an amount of about 1 ml is transferred to a 2 ml cryovial
32. The method according to any one of embodiments 11 to 30, wherein an amount of about 20 ml is transferred to a 50 ml cryobag.
33. The method according to any one of embodiments 11 to 32, wherein the batch of cell suspension in step a) is formulated in a cell culture spinner bottle, and wherein the cell culture spinner bottle is placed in a cooling apparatus according to any one of embodiments 1 to 10.
34. The method according to any one of embodiments 11 to 33, wherein the batch of cell suspension in step b) is subjected to agitation, such as shaking or stirring.
35. The method according to embodiment 34, wherein means for stirring is in the batch of cell suspension, such as a magnetic stirrer.
36. The method according to any one of embodiments 11 to 35, wherein at least a portion of the cell suspension remains in the batch of cell suspension maintained at said temperature during the time it takes to establish the cell bank.
37. The method according to any one of embodiments 11 to 36, wherein the method is carried out in a laminar flow workstation or a clean room.
38. Use of a cooling apparatus according to any one of embodiments 1 to 10 for the preparation of a cell bank from a batch of cell suspension, comprising maintaining the temperature of the batch of cell suspension at below 15° C., preferably below 12° C., preferably 0-12° C., preferably 0-11° C., preferably 0-10° ° C., more preferably 0-8° C., more preferably 2-8° C., more preferably 2-6° C., more preferably 3-5° C., even more preferably about 4° C.
39. The use according to embodiment 38, wherein the batch of cell suspension comprises stem cells or derived from stem cells.
40. The use according to embodiment 39, wherein the stem cells are human pluripotent stem cells (hPSCs).
41. A method of cooling a batch of cell suspension comprising the steps of:
   i. providing a cooling apparatus according to any one of embodiments 1 to 10,
   ii. loading a refrigerant into the first compartment of the cooling apparatus, wherein the temperature of the refrigerant is suitable for maintaining a temperature of the batch of cell suspension below 15° C.,
   iii. loading a flask containing the batch of cell suspension into the second compartment of the cooling apparatus, and
   iv. maintaining a temperature of the batch of cell suspension below 15° C.
42. The method according to embodiment 41, wherein the method is GMP compliant.
43. The method according to any one of embodiments 41 and 42, wherein the refrigerant is a cooling pack.
44. The method according to any one of embodiments 41 to 43, wherein the temperature of the refrigerant is from −20 to −80° C. when loaded into the first compartment.
45. The method according to any one of embodiments 41 to 44, wherein the batch of cell suspension is maintained at said temperature for at least one hour.
46. The method according to any one of embodiments 41 to 45, wherein the temperature of the batch of cell suspension is maintained at 0-15° C., preferably 0-12° C., preferably 0-10° C., preferably 0-8° C., more preferably 2-8° C., more preferably 2-6° C., more preferably 3-5° C., even more preferably about 4° C.

### EXAMPLES

The following are non-limiting examples for carrying out the invention.

### Example 1: Protocol for Establishing a Large Cell Bank

During preparation vials are precooled at 2-8° C. Gel packs and metal supports for the cooling apparatus are frozen at −20° C. overnight for at least 4 hours prior to cryopreservation.

Initially, hPSCs are formulated in STEM-CELLBANKER (SCB) cryopreservation medium. Cells at appropriate confluency are then photographed, and the cells are harvested and counted. Based on the total viable cell count obtained, the number of vials that can be filled with e.g. 1×10⁶ total viable cells per vial is determined. The calculated volume of cell suspension is then transferred to an appropriate sterile conical tube and kept cold. For small and large cell suspension volumes depending on the culture format is centrifuged at 300 g for 3 min and 5 min to pellet the cells. In the biosafety cabinet (BSC), the supernatant is aspirated, then the tube is tapped to loosen the cell pellet. The cell pellet is gently re-suspended with 1-5 mL of SCB. More SCB is added to bring the cell suspension to a volume yielding a concentration of e.g. 1×10⁶ cells/ml.

The cell suspension is transferred to a 1 L spinner flask and placed in a cooling apparatus according to the present invention. The pre-frozen cool packs and metal supports are inserted in the cooling apparatus. The cooling apparatus is placed with the cell suspension on a magnetic stirrer, ensuring that the cooling apparatus is centered on the stir plate. The mixing set to speed as appropriate. While filling as the volume reduces, the mixing speed may be changed accordingly.

The fill-it system (Sartorius Stedim Biotech), a bench-top automated cryovial processing system is used to automatically uncap, fill and recap the cryovials, 8-way single-use, sterile tube set to fill 48 tube rack is connected to the spinner flask liquid transfer port tube. The required number of cryovials is then filled with 1 mL fill volume using the systems peristaltic pump. Alternatively, the cryovials can also be manually filled using e.g. a serological pipette.

The cryovials are then transferred to a controlled rate freezer for cryopreservation. Once the target temperature has been reached, the cryovials placed on dry ice or directly to pre-cooled racks and transferred to the vapor phase of liquid nitrogen for long-term storage. Alternatively, a Mr. Frosty or BioCision CoolCell cryocontainer is used. Cryocontainer with filled cryovials is later placed in a −80° C. freezer for 4-48 hr. Subsequently the cryovials are transferred to the vapor phase of liquid nitrogen for long-term storage.

### Example 2: Comparison of Cell Viability at Different Temperatures

The present inventors have investigated the impact of longer exposure time with a cryoprotectant on hPSCs post-thaw (% viability, plating and recovery), i.e. samples were taken at specific time points and immediately cryopreserved, then later thawed were the viability of the cell sample was then assessed. Assessment of viability by Tryphan blue is well-described in e.g. Strober W. Trypan Blue Exclusion Test of Cell Viability. Curr Protoc Immunol. 2015; 111:A3.B.1-. A3.B.3. Published 2015 Nov. 2.

doi: 10.1002/0471142735.ima03bs111.

A comparison was carried out between hPSCs subjected to the method as described in Example 1 and a similar method wherein the cell suspension was not maintained at a temperature below 12° ° C. The viability was tested by thawing the cryopreserved cell samples and using the Tryphan blue exclusion method.

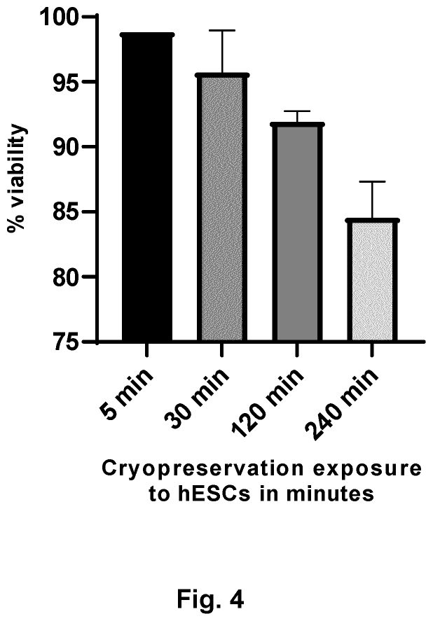

FIG. 4 shows viability by Tryphan blue exclusion method of hESCs over different exposure time to cryopreservation medium (specifically, STEM-CELLBANKER) at room temperature (approximately 20° C.) in two separate experiments. Clearly, the viability decreases the longer the hPSCs are in a cell suspension maintained at room temperature (RT). Data is presented in Table 1.

TABLE 1
Viability study at room temperature (% viability)

| 5 min | 30 min | 120 min | 240 min |
|---|---|---|---|
| 98.8 | 98 | 91.3 | 82.5 |
| 98.8 | 93.4 | 92.5 | 86.5 |

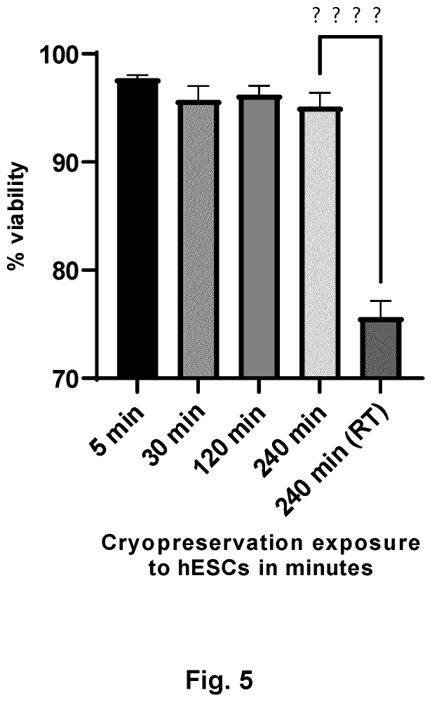

FIG. 5 shows the results where the cell suspension has been maintained at about 4° C. together with a comparative sample maintained at room temperature (RT). Data presented in Table 2 and statistical summary of comparison with samples taken at 240 minutes 4° C. and room temperature, respectively, presented in Table 3.

TABLE 2
Viability study with cells maintained
at approximately 2-8° C. (% viability)

| 5 min | 30 min | 120 min | 240 min | 240 min (RT) |
|---|---|---|---|---|
| 98 | 97.2 | 95.8 | 95.7 | 77.4 |
| 97.8 | 95.3 | 95.7 | 93.6 | 74.7 |
| 97.4 | 94.7 | 97.2 | 96 | 74.9 |

TABLE 3
ANOVA summary of viability study

| | |
|---|---|
| F | 197.6 |
| P value | <0.0001 |
| P value summary | **** |
| Significant diff. among means (P < 0.05)? | Yes |
| R squared | 0.9875 |

### Example 3: Measurement of the Temperature of the Cryopreservation Medium

In one experiment, the present inventors measured the temperature of the cryopreservation medium in a flask when placed in the cooling apparatus according to the present invention. Two temperature probes where used at the top and bottom of the flask, respectively. STEM-CELLBANKER was used as cryopreservation medium in a 1 L spinner flask with magnetic stirring and a medium volume of 850 ml. Refrigerant and metal plates were frozen prior to the experiment at −20° C.

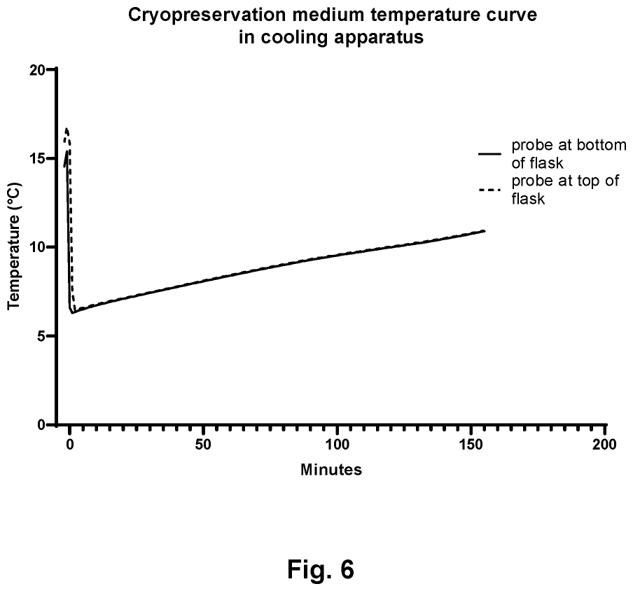

In FIG. 6 is shown the temperature measurement over time. Just prior to 0 minutes the STEM-CELLBANKER was added at a temperature of about 6° C. The refrigerant was not replaced during the experiment.

### Example 4: Comparison of Cell Viability of Differentiated Cells (hESC Derived Beta Like Cells) at Different Temperatures

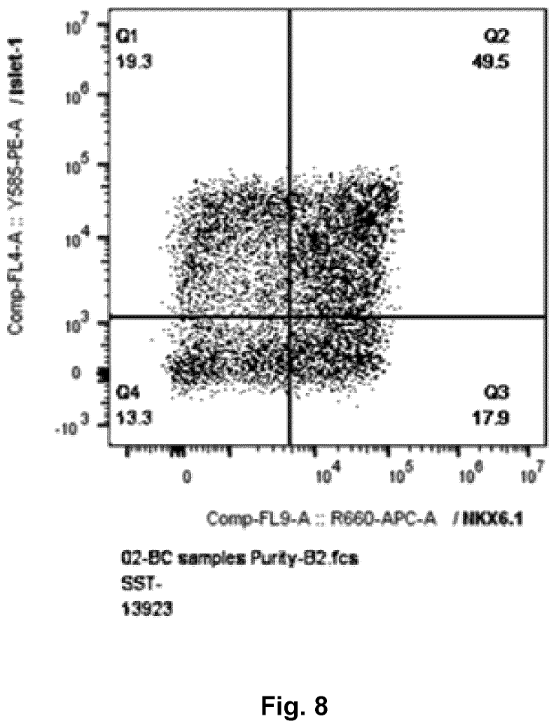

To test the holding time of immature beta cells, single cells at 100E6 VC/mL in StemCell Banker the following experiment was set up: Following the BC03 harvest from 1 L DASgip bioreactor using standard SC2BC differentiation protocol. FIG. 8 shows flow cytometry dot plots comparing percentage of cells expressing NKX6.1 and Islet-1 marker. 49.5% co-expresses the two markers denoting beta cells. Q0720882. 1.8 mL vials containing 1 mL of 100E6 VC/mL was stored at the following conditions before transfer to −80° C.:

TABLE 4
Room temperature storage

Time when put into −80° C.

| | 5 min | 30 min | 120 min | 240 min |
|---|---|---|---|---|
| # of 1 mL vials | 3 | 4 | 4 | 4 |

TABLE 5
4° C. temperature storage

Time when put into −80° C.

| | 5 min | 30 min | 120 min | 240 min |
|---|---|---|---|---|
| # of 1 mL vials | 2 | 4 | 4 | 4 |

All the vials are transferred to liquid nitrogen the next day. When thawed the following protocol was followed:

Preparation

| Step | Action |
|---|---|
| 1 | Warm 15 mL RPMI to 37° C. for each vial to thaw |
| 2 | Turn on thaw star |
| 3 | When media is ready add Rock Inhibitor (Y-27632, 5 mM): |
| | 1 μl/mL in the Biosafety Cabinet (BSC) |
| 4 | For each vial to thaw, prepare 3 × 15 mL tubes and one |
| | eppendorf tube: |
| | Tube A: 8 mL media for thawing |
| | Tube B: 5 mL media for rinsing and resuspension |
| | Tube C: Empty tube with 40 μm pluriStrainer mini |
| | Eppendorf tube: 950 μl media |
| 5 | Label all tubes with number according to order of thawing |

Thaw, straining and washing

| Step | Action |
|---|---|
| 1 | Thaw vial using ThawStar |
| 2 | With a p1000 pipette add 1000 μL of warm media to the cells, |
| | and carefully pipette up and down to thaw the last cells |
| 3 | Using the same pipette, transfer cells to Tube A containing |
| | 8 mL of media |
| 4 | Still using the same pipette wash cryovial with 1 mL media and |
| | transfer to cell suspension in Tube A |
| 5 | Spin cells at RT, 300 g, 3 min. Remove supernatant |
| 6 | Loosen the cell pellet by tapping the bottom of the centrifuge |
| | tube gently against the surface of the BSC |
| 7 | With a p1000 pipette carefully resuspend cells in 1 mL warm |
| | medium. Pipette 3-5 times up and down, until cells are fully |
| | resuspended. |
| 8 | Using the same pipette, strain the cell suspension through the |
| | cell strainer into Tube C. |
| 9 | Still using the same pipette, rinse Tube A with 1 mL media and |
| | run it through the cell strainer to wash the filter. |
| 10 | Mix the cell suspension and transfer 50 μl to the eppendorf |
| | tube containing 950 μl media. (1:20 dilution) |
| 11 | Count cells twice with NucleoCounter 202 |
| | Check that the cell count is within 0.5−10e6 total cells/mL |
| | (the nucleoCounter has decreased precision outside this range) |
| 13 | Note down the viability of the cells. |
| 14 | Count the cells a third time if the Viability of the two counts |
| | varies with more than 5% |

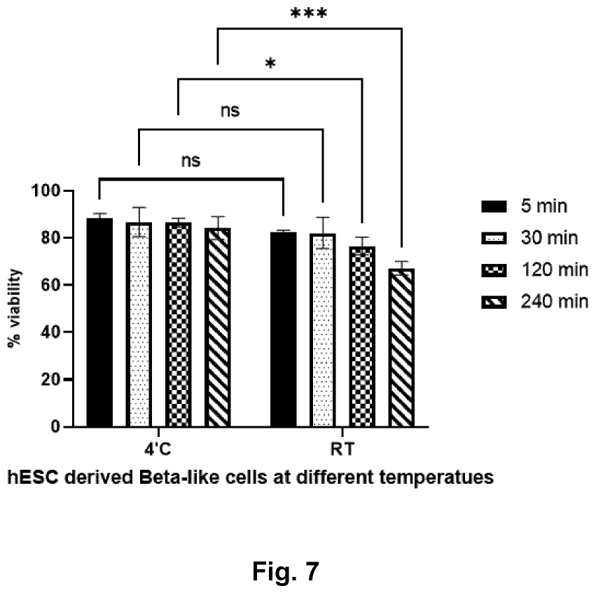

The results are presented in FIG. 7 showing % viability measured by NC-202 for different holding time of cryopreservation medium (Like, StemcellBanker) at room temperature and around 4 degrees Celsius. The average % viability drops from 82 to 66 at RT compared to 89 to 85 at 4° C. (5 min to 240 min). 6-8 vials thawed for each time point and by 3 different operators. 2 way ANOVA Sidak's multiple comparison test done to compare RT vs 4° C. (Table 6).

TABLE 6
2way ANOVA Sidak's multiple comparison test done to compare RT vs 4° C.

| \check{S}ídák's multiple | | | | | | |
|---|---|---|---|---|---|---|
| comparisons | | Predicted | | | | |
| test | | (LS) mean | 95.00% CI | Below | | Adjusted |
| 4° C.-RT | | diff. | of diff. | threshold? | Summary | P Value |
| 5 | min | 6.200 | −4.788 to 17.19 | No | ns | 0.4310 |
| 30 | min | 4.675 | −5.153 to 14.50 | No | ns | 0.5876 |
| 120 | min | 9.933 | 0.1056 to 19.76 | Yes | * | 0.0471 |
| 240 | min | 17.06 | 7.228 to 26.88 | Yes | *** | 0.0008 |

### Example 5: Cryopreservation of hESC Derived RPE Cells

E1C3 (NN GMP0050E1C3) cultured on iMatrix-511 (0.25 mg/cm2, Nippi) were differentiated to RPE cells as described in Petrus-Reurer, Sandra et al. “Molecular profiling of stem cell-derived retinal pigment epithelial cell differentiation established for clinical translation.” Stem cell reports vol. 17, 6 (2022): 1458-1475. doi: 10.1016/j.stemcr.2022.05.005.

To test the cryopreservation medium holding time in different temperature with RPE and how that impacts the viability of RPE. Cells were initially kept in Stem Cell Banker (at RT and 4° C. for following period of time mentioned below) before transfer into the −80° C. freezer.

RT: 60 min, 120 min and 240 min

4C: 10 min, 30 min, 60 min, 120 min and 240 min.

A positive control was prepared. This (0 min) constitutes cells that were transferred directly to the −80° C.

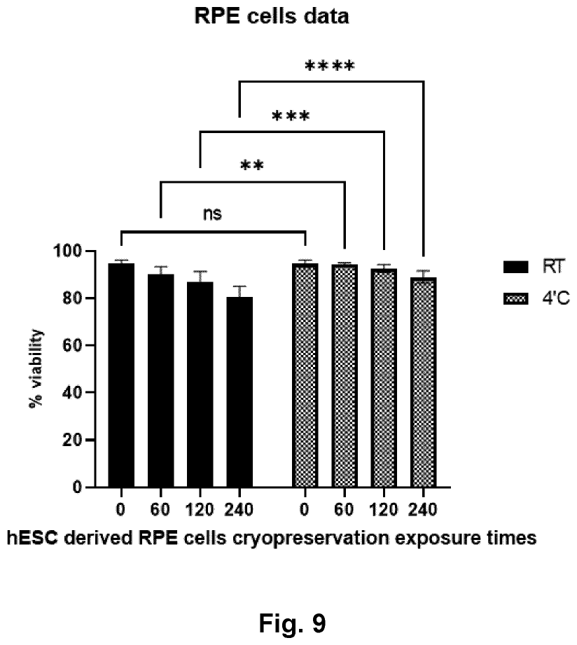

The results are presented in FIG. 9 showing % viability measured by NC-202 for different holding time of cryopreservation medium (Like, StemcellBanker) at room temperature and around 4 degrees Celsius. The % viability drops from 95 to 81 at RT compared to 95 to 89% at 4° C. (0 min to 240 min). 8-10 vials thawed for each time point and by 3 different operators. 2 way ANOVA Sidak's multiple comparison test done to compare RT vs 4° C. as shown in Table 7.

TABLE 7
2way ANOVA Sidak's multiple comparison test
in viability study of hESC derived RPE cells

| \check{S}ídák's multiple | | | | | |
|---|---|---|---|---|---|
| comparisons | Predicted | | | | |
| test | (LS) mean | 95.00% CI | Below | | Adjusted |
| RT-4° C. | diff, | of diff, | threshold? | Summary | P Value |
| 0 | 0.000 | −3.413 to 3.413 | No | ns | >0.9999 |
| 60 | −4.500 | −8.018 to −0.9823 | Yes | ** | 0.0067 |
| 120 | −5.500 | −8.826 to −2.174 | Yes | *** | 0.0003 |
| 240 | −8.380 | −11.62 to −5.142 | Yes | **** | <0.0001 |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for establishing a cell bank comprising:
a. providing (i) a batch of cell suspension comprising stem cells or stem cell-derived cells and (ii) a cryopreservation medium,
b. maintaining the batch of cell suspension in the cryopreservation medium at a temperature below 15° C., and
c. transferring an amount of the cell suspension from the batch of cell suspension into one or more storage containers to establish the cell bank,
wherein establishing the cell bank takes at least 30 minutes.

2. The method according to claim 1, wherein the amount of the batch of cell suspension is transferred to at least 10 storage containers.

3. The method according to claim 1, wherein the temperature is maintained below 8° C.

4. The method according to claim 1, wherein the temperature is maintained at about 4° C.

5. The method according to claim 1, where the temperature is maintained above the freezing point of the cell suspension.

6. The method according to claim 1, wherein the temperature is maintained above 0° ° C.

7. The method according to claim 1, wherein establishing the cell bank takes at least one hour.

8. The method according to claim 1, further comprising:
d. freezing each of the storage containers immediately following transfer of the amount of the cell suspension from the batch of cell suspension.

9. The method according to claim 1, wherein the amount of cell suspension transferred into each storage container is from 0.25 to 1000 ml.

10. The method according to claim 1, wherein the volume of the batch of cell suspension is at least 25 ml.

11. The method according to claim 1, wherein the stem cells are pluripotent stem cells (PSCs) and wherein the stem cell-derived cells are selected from the group consisting of beta like cells, cardiomyocytes, and retinal pigment epithelium cells (RPE cells).

12. A cooling apparatus for cooling a batch of cell suspension during preparation of a cell bank comprising: a housing comprising an outer wall, a first compartment adapted to receive a refrigerant, and a second compartment adapted to hold a container with the batch of cell suspension, wherein the first compartment and second compartment are separated by a heat conducting element, wherein the refrigerant is capable of cooling the batch of cell suspension when the refrigerant is loaded into the first compartment and the container with the batch of cell suspension is loaded into the second compartment.

13. A method of cooling a batch of cell suspension comprising the steps of:
i. providing a cooling apparatus according to claim 0,
ii. loading a refrigerant into the first compartment of the cooling apparatus, wherein the temperature of the refrigerant is suitable for maintaining a temperature of the batch of cell suspension below 15° C.,
iii. loading a flask containing the batch of cell suspension into the second compartment of the cooling apparatus, and
iv. maintaining a temperature of the batch of cell suspension below 15° C.

14. The method according to claim 13, wherein the temperature of the batch of cell suspension is maintained at 0-15° C.

15. (canceled)
